(19) United States
(12) Patent Application Publication
(10) Pub. No.: US 2011/0009808 A1
(43) Pub. Date: Jan. 13, 2011

(54) Multi needle apparatus for treating total volume of skin lesions by intralesional injection method

(75) Inventor: Khalid M AlGhamdi, Riyadh (SA)

Correspondence Address: STEVEN A. NIELSEN;ALLMAN & NIELSEN, P.C., 100 Larkspur Landing Circle, Suite 212, LARKSPUR, CA, 94939, US

(73) Assignee: KING SAUD UNIVERSITY, Riyadh (SA)

(21) Appl. No.: 12/498,917

(22) Filed: Jul. 7, 2009

(51) Int. Cl. A61M 37/00

(52) U.S. Cl. 604/22

(57) ABSTRACT

Viral warts are penetrated with a multiple solid needle apparatus which applies a bleomycin medication to the total volume of the wart. The disclosed method and apparatus provides a one step or two step procedure to quickly and efficiently penetrate all parts of a wart and evenly apply the bleomycin into the wart. Multiple solid needles are uniformly and symmetrically spaced and secured within a base unit. A syringe is used to press the base unit into a wart as well to deliver the bleomycin solution.

### BACKGROUND OF THE INVENTION

(1) Field of the Invention

The invention generally relates to means and methods of treating warts. More particularly, the invention relates to a multiple needle apparatus and intralesional method of delivering bleomycin medication to all parts of a wart.

(2) Description of the Related Art

Viral warts is a common skin disease that affects the appearance and psychology of the patient. Some types of viral warts are considered resistant to conventional treatments and if not treated effectively such warts proliferate and become even more difficult to treat. Within the known related art, there are numerous treatment options for viral warts which demonstrate variable degrees of effectiveness (Sollitto et al., 1989). Intralesional treatment with bleomycin is one of the most effective methods of treatment and has been used since 1970 (Lewis and Nydord, 2006). Bleomycin kills the virus that causes the wart through binding to its DNA and inhibiting its proliferation (Saitta et al., 2008).

Bleomycin was originally injected below the dermis, i.e. under the wart, but this method was ineffective, since the medication did not reach all parts of the wart (Bunney et al., 1984). Subsequently, an improved method of medication delivery was achieved by using a Tuberculin needle. Puncturing the surface of the wart was used as a delivery method to allow Bleomycin to penetrate the wart.

Later, the method of using multiple punctures was described(Shelley and Shelley, 1991). The wart was first injected with a local anesthesia (2% Lidocaine) and then a drop of the Bleomycin liquid would be placed on the surface of the wart. Punctures were made to create holes that reach the base of the wart. About 50 punctures are made in a space of 5 mm², the wart is then covered with a bandage. Three weeks later the wart becomes necrosed and falls off. The treatment might need to be repeated if some parts of the wart stay visible. Later, a dermatography technique was described. Dermatography is a method of tattooing that allows a substance to be injected at a controlled depth. Van der Velden et al. used the Van der Velden derma-injector (10,000 punctures produced in the skin per minute)to inject bleomycin in warts. This technique provides an equal distribution of the bleomycin solution throughout the wart and results in less pain (Van der Velden et al., 1997).

The known related art presents at least four shortfalls:

1. Using a single needle, a large number of individually pressed punctures are required to attempt to disperse the medication to all areas or parts of the treated wart. A large number of individual punctures performed in series presents a time consuming and laborious endeavor.

2. Using a single needle and multiple individual punctures presents a probability that all affected areas of the wart will not come into contact with the medication.

3. The traditional single needle method may employ a hollow needle to deliver medication at various depths. The use of a hollow needle in a multi puncture procedure is prone to blockage by wart debris entering the needle. The danger of needle mishandling or needle sticks is great as the related art requires several needle replacements during a typical application of wart medication.

4. The Van der Velden derma-injector requires a separate source of electricity, mechanical maintenance and a skilled medical technician to avoid mishaps. Moreover, the Van der Velden derma-injector is often cost prohibitive and unnecessarily intimating to patients.

### BRIEF SUMMARY OF THE INVENTION

The present invention overcomes shortfalls in the related art by presenting an effective and efficient method of delivering medication to multiple interior areas of a wart. The disclosed technique and multiple needle apparatus present an unobvious combination of components and procedures to achieve unexpected results and advantages in the field of wart treatment. The disclosed apparatus and method includes, but are not limited to the following advantages over the related art.

1. The short fall of multiple penetrations in series is overcome by the present invention's configuration of multiple needles and means of medication delivery at multiple depths within a wart. In the present invention, one or two presses of 50 needles configured within the disclosed apparatus replaces the method of the prior art where 80 to 100 individual penetrations are required. The present invention provides an unexpected time savings of 50% to 80% as compared to the known related art. A wart may range in size from one square centimeter to several square centimeters, thus making prior methods more time consuming when faced with a larger wart.

2. The short fall in the related art of missing areas within a wart is overcome by the use of a grid of needles with an optimal diameter and optimal spacing. The disclosed configuration of needles provides a controlled and refined intralesional disbursement of wart mediation. After one pressing of the disclosed apparatus, a visual pattern of needle placement is evident which allows for a quick and convenient alignment of the apparatus for an optional second wart treatment. The equidistant nature of the disclosed needle configuration lessens the probability of missing wart areas during treatment.

3. The use of solid needles and a top loading medication system overcomes the related art's shortfall of needle clogging. Unlike the related art, the disclosed system of solid needles is not prone to failure by the problem of wart debris interfering with the flow of wart medication. A void within the base structure above the needle gird provides means of allowing wart medication to evenly flow to the surface of the wart.

4. The uniform configuration and spacing of the multiple needles within the disclosed apparatus insures that a patient will receive a quick treatment of one or two pressings.

Furthermore, the present invention overcomes shortfalls in the art by providing means to use a less concentrated dosage of wart medication as found in the related art. The uniform configuration of multiple needles and use of solid needles provides a more efficient delivery system as opposed to the related art. A decrease in dosage will lead to a decrease in unwanted side effects and to a decrease in treatment costs. The disclosed configuration of needles provides an economical format for needle production as the needles may be cast from a single pouring of needle material.

### BRIEF DESCRIPTION OF THE DRAWINGS

FIG. 1 is side view of a single hollow head needle of the related art.

FIG. 2 is a side view of a multiple needle apparatus consistent with the principles of the disclosed invention.

FIG. 3 is a side view of a syringe affixed to the top of the disclosed multiple needle apparatus.

FIG. 4 is a cross sectional view of the needles of the disclosed apparatus.

FIG. 5 is a cross sectional view of a horizontal wart surface after puncturing with the disclosed apparatus.

### REFERENCE NUMBERS

5 is a hollow needle of the related art.

10 is a base structure that holds the needles 30 and contains a fluid opening 20 and fluid reservoir 21.

20 is an opening used to allow wart treatment medication or other fluid to travel between the needles and to the wart surface 21.

21 is a reservoir contained within the base structure 10. In the preferred embodiment, the reservoir 21 holds one or more drops of wart medication.

30 is a group of solid needles.

40 is a syringe attached to a base structure 10.

50 is a cross sectional representation of a needle from a group 30 of needles.

60 is a dark round area upon a wart that has been treated by a first pressing of the disclosed apparatus.

61 is a clear round area upon a wart that has been treated by a second pressing of the disclosed apparatus.

### DETAILED DESCRIPTION OF EMBODIMENTS OF THE INVENTION

The present invention is sometimes described here as the “disclosed method and apparatus”, “multiple needle apparatus”, and/or the “disclosed invention”. The upward direction means at a direction away from the skin surface. The downward direction means the direction into the skin of the patient.

In the following description, for purposes of explanation, numerous specific details are set forth in order to provide a thorough understanding of the invention. It will be apparent, however, to one skilled in the art that the invention can be practiced without these specific details. In other instances, structures and devices are shown in block diagram form in order to avoid obscuring the invention.

The reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment nor are separate alternative embodiments mutually exclusive of other embodiments.

In the following detailed description of embodiments of the invention, reference is made to the accompanying drawings in which like references indicate similar elements, and in which is shown by way of illustration specific embodiments in which the invention may be practiced. These embodiments are described in sufficient detail to enable those skilled in the art to practice the invention, and it is to be understood that other embodiments may be utilized and that logical, mechanical, electrical, functional, and other changes may be made without departing from the scope of the present invention. The flowing detailed description is, therefore, not to be taken in a limiting sense.

The description, which follows, and the embodiments described therein, are provided by way of illustration of an example, or examples of particular embodiments of the principles of the present invention. These examples are provided for the purposes of explanation, and not of limitation, of those principles of the invention. In the description, which follows, like parts are marked throughout the specification and the drawings with the same respective reference numerals. The drawings are not necessarily to scale and in some instances proportions may have been exaggerated in order to more clearly depict certain features of the invention.

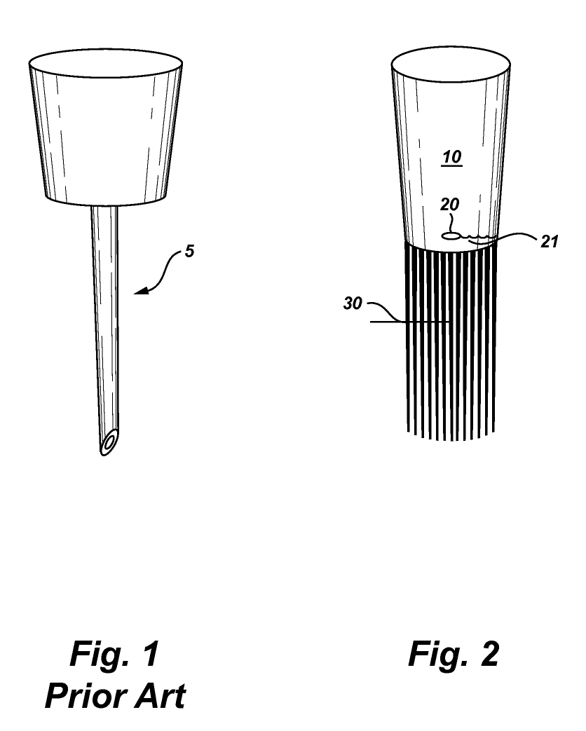

Referring to FIG. 1, a hollow hypodermic needle of the prior art is shown. In the related art, the use of such a needle would lead to various problems, including the accumulation of wart material in the inner hollow section of the needle. The accumulation of wart debris within the needle would lead to a blockage of fluid flow or an unpredictable volume of fluid leaving the needle.

Referring to FIG. 2 a base unit 10 has means of attachment to a syringe and means of holding fluid within a reservoir 21 contained within the base unit. In the lower center of the base unit, a void 20 allows fluid to drip past the needles and on to a wart located below the needles 30. The use of a void 20 and the storage of medicine within the reservoir 21 provide unexpected results by placing the medicine in the exact middle area of the needle configuration. The center void allows for one handed use of the apparatus and significantly reduces treatment time, as compared to applying wart medicine with a separate syringe or separate fluid delivery system.

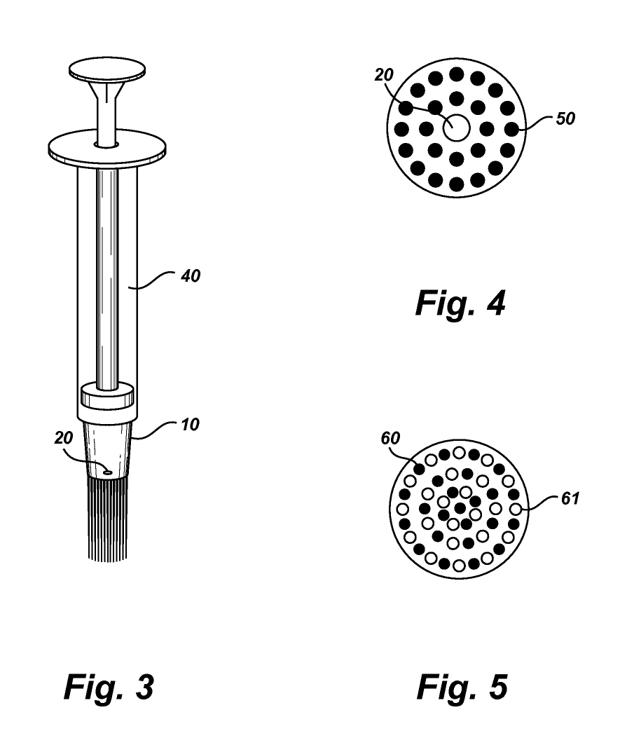

Referring to FIG. 3 a syringe 40 has been attached to the base unit 10 which allows for pressure to be used to move fluid through void 20.

Referring to FIG. 4 a bottom plan view of the base unit is presented wherein needles 50 surround center void 20.

Referring to FIG. 5, a top plan view of a wart treated once as shown with dark marks 60 and treated with a second press as shown by light marks 61.

The invention includes, but is not limited to the following items:

[Item 1.] A method of treating viral warts, the method comprising the steps of:

a) applying wart medication to a wart by pressing the wart medication through a center void found within a base unit;

b) allowing the wart medication to spread over the surface of the wart;

c) using a multiple needle apparatus, the apparatus having a concentric configuration of needles with the center void placed in the center of the needles, to press the wart medication into the wart;

[Item 2] The method of item 1 wherein a second press of the multiple needle apparatus occurs after the first press of item 1 and wherein the multiple needle apparatus is moved such that new areas of the wart are treated.

Item 3 A system for use in treating viral warts, the system comprising:

a) a plurality of solid needles concentrically attached to a base unit;

b) a void within the bottom center section of the base unit, such that the edges of the void do not contact the needles;

c) an interior space within the base unit with means of holding wart medicine; and

d) the top section of the base unit having means to integrate with a syringe such that the syringe may apply pressure to the fluid and cause the fluid to flow to a wart.

What is claimed is:

1. A method of treating viral warts, the method comprising the steps of:
a) applying wart medication to a wart by pressing the wart medication through a center void found within a base unit, with the base unit having a reservoir to store wart medication and with the base unit attached to multiple solid needles;
b) allowing the wart medication to spread over the surface of the wart; and
c) using a multiple needle apparatus, the apparatus having a concentric configuration of solid needles with the center void placed in the center of the solid needles, to press the wart medication into the wart.

2. The method of claim 1 wherein a second press of the multiple needle apparatus occurs after the first press of claim 1 and wherein the multiple needle apparatus is moved such that new areas of the wart are treated.

3. A system for use in treating viral warts, the system comprising:
a) a plurality of solid needles concentrically attached to a base unit;
b) a void within the bottom center section of the base unit, such that the edges of the void do not contact the needles;
c) an interior space within the base unit with means of holding wart medicine; and
d) the top section of the base unit having means to integrate with a syringe such that the syringe may apply pressure to the fluid and cause the fluid to flow to a wart.
